(19) United States
(12) Patent Application Publication
(10) Pub. No.: US 2026/0306827 A1
(43) Pub. Date: Oct. 8, 2026

(54) IONTOPHORESIS PHARMACEUTICAL DELIVERY SYSTEM

(71) Applicants: The University of Melbourne, Melbourne (AU); Remagine Labs, Inc., Austin, TX (US)

(72) Inventors: Efstratios SKAFIDAS, Melbourne (AU); Neil Patrick KELLY, Austin, TX (US); Steven Grant DUVALL, Manly (AU)

(21) Appl. No.: 19/484,435

(22) PCT Filed: May 24, 2024
(86) PCT No.: PCT/AU2024/050540

§ 371 Date: Nov. 14, 2025

Related U.S. Application Data
(60) Provisional application No. 63/469,047, filed on May 25, 2023.

(51) Int. Cl. A61N 1/04; A61N 1/02; A61N 1/08; A61N 1/32

(52) U.S. Cl. CPC A61N 1/0448; A61N 1/025; A61N 1/0496; A61N 1/08; A61N 1/325

(57) ABSTRACT

This disclosure relates to an iontophoresis pharmaceutical delivery system comprising an array of individually addressable pharmaceutical delivery sub-systems. Each of the sub-systems comprises a delivery electrode that is insulated from electrodes of other sub-systems and a reservoir for holding the pharmaceutical to be delivered. A control circuit generates an electrical control signal and provides the electrical control signal to the delivery electrode of one the sub-systems to deliver a pharmaceutical transdermally by iontophoresis. With this array one delivery sub-system can be activated until its corresponding reservoir is depleted before activating another sub-system. Therefore, the amount of the pharmaceutical that has been delivered is known at least down to the number of reservoirs that have been depleted. Further, it is possible to simultaneously activate multiple sub-systems to increase the dose rate and to load different drug formulations into each sub-system thereby delivering multiple drugs simultaneously.

### CROSS-REFERENCE TO RELATED APPLICATIONS

The present application claims priority from U.S. of America Provisional Patent Application No 63/469,047 filed on 25 May 2023, the contents of which are incorporated herein by reference in their entirety.

### TECHNICAL FIELD

This disclosure relates to an iontophoresis pharmaceutical delivery system.

### BACKGROUND

Iontophoresis delivers a medicine or other chemical through the skin, colloquially referred to as “an injection without a needle”. Iontophoresis is non-invasive and different from passive transdermal patches, which do not rely on an electric field or current. During iontophoresis a delivery system drives a substance, usually a medication or bioactive agent, transdermally by electromotive force, through the skin. A small electric current is applied to an iontophoretic chamber adhered to the skin, containing a charged active agent and its solvent vehicle. Another chamber or a skin electrode carries the return current. The positively charged electrode, called the anode, repels a positively and attracts a negatively charged chemical species into or from the skin, whereas the negatively charged electrode, called the cathode, repels a negatively and attracts positively charged species into or from the skin.

There are a range of problems associated with existing iontophoresis devices. These include poor control over the administered amount of the substance, degradation of the electrodes over time, creation of unwanted chemical species as a result of the electrochemical reactions and discomfort and in some cases skin damage in patients due to the applied electrical voltage. Size and weight are also a problem with existing devices, which prevent them from being used as a long-term pharmaceutical delivery vehicle.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

An iontophoresis pharmaceutical delivery system comprises:
an array of individually addressable pharmaceutical delivery sub-systems, each of the individually addressable pharmaceutical delivery sub-systems comprising a delivery electrode that is insulated from electrodes of other pharmaceutical delivery sub-systems and a reservoir for holding the pharmaceutical to be delivered;
a control circuit configured to generate an electrical control signal and provide the electrical control signal to the delivery electrode of one or more of the individually addressable pharmaceutical delivery sub-systems to deliver a pharmaceutical transdermally by iontophoresis.

It is an advantage that the array of individually addressable pharmaceutical delivery sub-systems provides for improved control of the pharmaceutical delivery because one delivery sub-system can be activated until its corresponding reservoir is depleted before activating another sub-system. Therefore, the amount of the pharmaceutical that has been delivered is known at least down to the number of reservoirs that have been depleted. Further, it is possible to simultaneously activate multiple sub-systems to increase the dose rate and to load different drug formulations into each sub-system thereby delivering multiple drugs simultaneously.

In some embodiments, the reservoir is separated from reservoirs of other pharmaceutical delivery sub-systems.

In some embodiments, the system is integrated into an adhesive patch for transdermal application.

In some embodiments, the system comprises an integrated battery.

In some embodiments, the control circuit comprises a signal generator to generate a signal that delivers electrical charge to the delivery electrode.

In some embodiments, the signal generator comprises a current source.

In some embodiments, the control circuit is further configured to control the signal generator to generate a biphasic signal to reduce or revert degradation of electrodes or build-up of undesired chemical species.

In some embodiments, the control circuit comprises a switch matrix to selectively connect the one or more of the individually addressable pharmaceutical delivery sub-systems to the signal generator.

In some embodiments, the system further comprises an embedded processor and program memory to store a control program, the system comprising the processor and the program memory being integrated into a transdermal patch, the control program causing the processor to control the signal generator.

In some embodiments, the embedded processor or the control circuit is configured to transmit or receive data using wireless communications.

In some embodiments, the system is further configured to transmit data to another device to store and interpret measurements, a pharmaceutical administration profile, and patient data.

In some embodiments, the system is configured to receive an authorisation from an external device for the commencement of administration of the pharmaceutical based on patient parameters including one or more of patient identity, patient condition, previously administered dose or response to the pharmaceutical or correctly applied patch.

In some embodiments, the system comprises a return electrode.

In some embodiments, the return electrode is provided with a reservoir for a charge balancing fluid.

In some embodiments, each sub-system comprises a respective return electrode and reservoir for a charge balancing fluid.

In some embodiments, each sub-system comprises a reference electrode to provide a stable electrical potential interface.

In some embodiments, each sub-system comprises the delivery electrode, a return electrode and a reference electrode.

In some embodiments, the control circuit is configured to selectively direct the control signal to one of the multiple sub-systems based on the measurement from the reference electrode.

In some embodiments, the controller is configured to measure an amount of current or charge delivered over time to estimate an amount of the pharmaceutical that has been delivered.

In some embodiments, the system is configured to measure an impedance between two of the electrodes to detect a condition comprising one or more of: the system is applied to skin to enable delivery of the pharmaceutical, the system has been detached from the skin, or, the system has been damaged.

In some embodiments, the system is further configured to provide an indication to a user of the detected condition and/or select electrodes that are applied to the skin to enable delivery of the pharmaceutical and deactivate electrodes that have been detached or damaged.

In some embodiments, the electrode is coated with an ion-transducing material.

In some embodiments, the electrodes are coated with materials that increase charge injection into the hydrogel.

In some embodiments, the electrodes are coated with materials to minimise the oxidation or reduction of chemical species that are present in the reservoir.

In some embodiments, the electrodes are designed to minimise electrolysis of water.

In some embodiments, the system is integrated into a patch comprising an adhesive for fixation on the skin of a patient.

In some embodiments, the adhesive for fixation on the skin of a patient is integrated into the reservoir.

In some embodiments, the system comprises a polymer on a skin contact surface of the patch to provide protection of the skin contact surface before application, and the polymer is degradable on application to the skin to create a direct contact between the patch and the skin.

In some embodiments, the system comprises a polymer skin interface being a membrane that is permeable to the pharmaceutical.

In some embodiments, the system comprises a membrane to separate the reservoir into multiple sections where each section may contain different substances.

In some embodiments, the pharmaceutical is dissolved using a solvent and incorporated into a hydrogel that is placed in a well that comprises at least one electrode.

In some embodiments, different wells contain different pharmaceuticals and the system is configured to select which of the pharmaceuticals to administer.

In some embodiments, the hydrogel forms a hydrogel matrix comprising an ionic substance to assist with pharmaceutical transport through the skin.

In some embodiments, the well and/or hydrogel matrix incorporates one or more chemical skin permeators.

In some embodiments, the system further comprises a mechanical skin permeator to improve transport of the pharmaceutical through the skin.

In some embodiments, for non-polar pharmaceuticals, molecules with both a polar and non-polar ends are present in the sub-systems and the molecules interact with the non-polar pharmaceutical to increase susceptibility to an electric filed.

In some embodiments, the pharmaceutical comprises a nanoparticle that is tagged with an ionic tag.

In some embodiments, the system comprises a reverse iontophoresis sub-system to measure a blood or interstitial fluid concentration of the pharmaceutical and the system is configured to adjust the electrical control system based on the measured blood concentration.

A method for delivering a pharmaceutical through iontophoresis comprises:
selecting one of multiple pharmaceutical delivery sub-systems each comprising a delivery electrode; and
applying an electrical control signal to the delivery electrode of the selected pharmaceutical delivery sub-system to deliver the pharmaceutical transdermally by iontophoresis.

### BRIEF DESCRIPTION OF DRAWINGS

FIG. 1 illustrates an iontophoresis pharmaceutical delivery system.

FIG. 2 illustrates a cross-section of a delivery sub-system.

FIG. 3 illustrates an example biphasic pulse.

FIG. 4 illustrates a control circuit.

FIG. 5 illustrates a method for delivering a pharmaceutical through iontophoresis.

### DESCRIPTION OF EMBODIMENTS

This disclosure provides an iontophoresis pharmaceutical delivery system that comprises an array of electrodes and corresponding reservoirs of a pharmaceutical that may be embedded in a polymer or gel in addition to other chemicals that may assist with skin permeation or stability of the pharmaceutical or adhesion to the skin. There are a number of advantages in such a system that will be described in more detail below. In general, the system provides more reliable control of the amount of pharmaceutical that is delivered and a reduction in unwanted chemical species and a reduction in electrode degradation in addition to further advantages described below. In example systems where the reservoirs include adhesive into the polymer or gel mixture, all but a small portion of the system area can be devoted to pharmaceutical delivery. This is an advantage over other devices that face a trade-off between how much of the device area is devoted to substance delivery versus the area used to adhere the device to the skin.

It is noted that an “array” as used herein, refers to a general collection of similar things arranged side-by-side. So in this instance, the array is a collection of multiple electrodes that may each be contained in a respective delivery sub-system as described below. As such, the array may be an ordered arrangement, such as a rectangular array of m×n sub-systems or a linear array, but may also be unordered, such as a random array or an array with a different ordering, such as a hexagonal or triangular array.

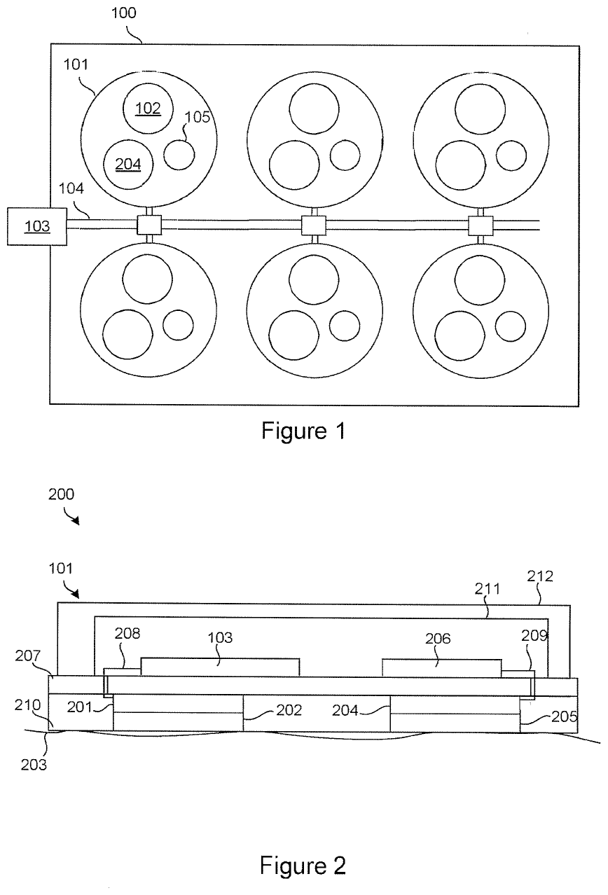

FIG. 1 illustrates an iontophoresis pharmaceutical delivery system 100 according to an embodiment. Delivery system 100 comprises an array of 3×2 individually addressable pharmaceutical delivery sub-systems, such as sub-system 101. Each of the individually addressable pharmaceutical delivery sub-systems, including sub-system 101 comprises a delivery electrode 102 that is insulated from electrodes of other pharmaceutical delivery sub-systems and a reservoir (not shown) for holding the pharmaceutical in liquid, polymer or gel milieu with other chemicals to be delivered and may also contain adhesive to adhere to the skin. The reservoir is separated from reservoirs of other pharmaceutical delivery sub-systems.

Delivery system 100 further comprises a control circuit 103, which may be a microprocessor, field programmable gate array, application specific integrated circuit or other type of circuit and may incorporate a wireless communications system. Control circuit 103 is configured, such as by way of program code stored on program memory or by way of hardware design, to generate an electrical control signal 104 and provide the electrical control signal to the delivery electrode 102 of one or more of the individually addressable pharmaceutical delivery sub-systems 101 to deliver a pharmaceutical transdermally by iontophoresis.

The array of individually addressable pharmaceutical delivery sub-systems 101 provides for improved control of the pharmaceutical delivery because one delivery sub-system can be activated until its corresponding reservoir is depleted before activating another sub-system. Therefore, the amount of the pharmaceutical that has been delivered is known at least down to the number of reservoirs that have been depleted. Further, it is possible to simultaneously activate multiple sub-systems to increase the dose rate and to load different drug formulations into each sub-system thereby delivering multiple drugs simultaneously.

FIG. 2 illustrates a cross-section 200 of delivery sub-system 101. As described above, delivery sub-system 101 comprises an electrode (or combination of electrodes) 201 and a drug reservoir 202. The drug reservoir 202 is proximal to skin 203 surface of the patient or user and may be in direct contact or in fluidic communication with the skin surface 203 so that the pharmaceutical can pass from the reservoir 202 into the skin surface 203. That is, there may be a permeable membrane or other layer between reservoir 202 and skin surface 203 but that layer is permeable to the pharmaceutical. There may also be multiple reservoirs between the electrode and the skin separated by various membranes. In this case, each reservoir may be comprised of a different combination of pharmaceutical formulation, and other chemicals or adhesives.

Sub-system 101 further comprises a return electrode 204 with a reservoir 205 for a charge balancing fluid. In some embodiments, each sub-system 1012 comprises a respective return electrode 204 and reservoir 205 for a charge balancing fluid. In other embodiments, multiple sub-systems 101 share a return electrode 204 and reservoir 205. The charge balancing fluid may be a salt and may be NaCl in a water solution. In operation, the electrode 201 provides positively or negatively charged ions into the skin and the return electrode 204 provides ions of the opposite polarity into the skin. Therefore, there is less build-up of charged particles in the skin. In one example, the electrode 201 operates as an anode and the return electrode 204 operates as a cathode, noting that the polarity may be reversed for other applications.

### Reference Electrode

In some examples, sub-system 101 further comprises a reference electrode 105 (not shown in FIG. 2) to provide a stable fluid electrode potential. The reference electrode 105 can also provide a measurement of the electrode-skin interface or impedance between the electrode and the skin. Advantageously, the reference electrode is located closely to the electrode 102 and return electrode 204 so that the measurement on the reference electrode 105 provides a reasonably accurate indication of the interface condition of the electrode 102 and return electrode 204. In other words, if the reference electrode 105 provides a measurement that indicates good skin contact, it can be assumed that the skin contact is also good for electrode 102 and return electrode 204.

In one example, each sub-system comprises the delivery electrode 102, return electrode 204 and a reference electrode 105. So the number of sub-systems is the same as the number of supply electrodes, returns electrodes and reference electrodes. This means for n sub-systems there are n*3 electrodes. It is also possible to have a system with a single return and reference electrode, in which case the number of electrodes would be n+1 or n+2. As show in FIG. 1, the area of the electrodes can be different, or the area of supply electrode 102 and return electrode 204 are identical whereas the reference electrode 105 has a smaller area because it does not need to hold any liquid in a reservoir and it does not need to deliver that liquid through the skin.

The control circuit 103 may measure an impedance between two of the electrodes, e.g. 102 and 204, to detect a condition of the electrodes. That is, circuit 103 may measure the impedance between those two electrodes and then detect the condition based on the impedance, such as whether the impedance satisfies a threshold condition. The conditions may be that the system is applied to skin to enable delivery of the pharmaceutical (i.e. there is a good contact, low impedance <50 Ω). The condition may also be that the system has been partially or wholly detached from the skin, or, the system has been damaged (i.e. there is a bad contact, high impedance >50 Ω).

In a further example, control circuit 103 selectively directs the control signal to one or more of the multiple sub-systems 101 based on the measurement from the reference electrode. That is, control circuit 103 selects one or more sub-systems for which measurement through the respective reference electrode indicates a good connection. Control circuit 103 may monitor and/or store the fill level of reservoir 202 and only select sub-systems with a full reservoir 202 or sub-systems with non-empty reservoir 202.

### Method of Fabrication

In one example, the system 100 is integrated into an adhesive patch for transdermal application. This integration means the control circuit 103 as well as sub-systems 101 and control signals 104 are integrated within the patch. Therefore, no further components are required and no further components need to be connected to the patch to commence application, which is why the patch may also integrate a battery 206. The method for manufacturing the system 100 commences by providing a substrate and creating the required metal layers on the substrate as well as making vias 208/209, which provide an electrical connection between the top side of substrate 207 to the bottom side of substrate 207. The metal layers comprise the signal lines 104 as well as electrodes 201 and 204 and may include an antenna. Next, the reservoirs 202 and 205 are created and incorporated into a membrane 210 or adhesive laminate or directly onto a protective liner. Further, the control circuit 103 and the battery are added 206, noting that there may only be one control circuit 103 and one battery 206 for the entire system 100. Finally, the top side of the substrate 207 is encapsulated in an encapsulating layer 211, such as epoxy cured with ultraviolet (UV) light and covered with an encapsulation foil or backing 212.

### Control Signals

It is noted that the sub-systems are individually addressable and the control signal 104 may be distributed over a switch matrix, which means that the control circuit 103 has one output for the delivery electrode 102 and one output for the return electrode 204 and one input for the reference electrode 105. The switch matrix then selectively connects these outputs and input to the respective electrodes of the selected one or more sub-system 101. It is noted that multiple sub-systems can be connected in parallel to increase the rate of application.

The switch matrix can switch to a different sub-system when the first selected sub-system has a depleted reservoir or in case the reference electrode 105 indicates a high impedance skin interface. The switch matrix can “scan” through other sub-systems by connecting the controller to one sub-system at a time until a sub-system with a good skin connection is found.

In some embodiments, the control circuit 103 comprises a signal generator that is controlled by the control circuit 103. The signal generator may be implemented as a current source that is controllable by the control circuit 103. For example, the control circuit 103 may output a multi-bit digital signal that the current source converts to a reference current feeding a current mirror, that then automatically regulates the voltage, through a power transistor circuit, to enforce the set reference current through the skin interface and thereby deliver a controllable amount of pharmaceutical into the skin.

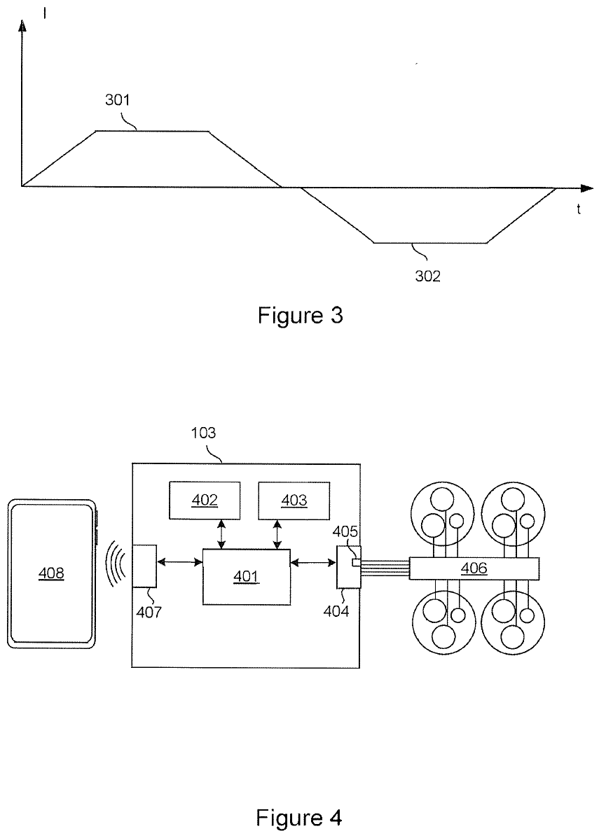

In some examples, the signal generator generates a biphasic signal to reduce or revert degradation of electrodes or build-up of undesired chemical species. FIG. 3 illustrates an example biphasic pulse, where the figure shows the current between the delivery electrode 102 and the return electrode 204. The pulse has a positive current period during which the positively charged pharmaceutical is delivered to the skin by the delivery electrode 102 and the current balancing liquid is delivered to the skin by the return electrode 204. During this positive current period, the electrodes may experience a degree of corrosion or oxidation and undesirable species may be generated, such as by electrolysis. This may lead to the accumulation of hydrogen peroxide or other unwanted species, which may cause, skin irritation, immune response or scarring. A negative current period 302 is therefore applied between the electrodes to at least partly reverse the effects of corrosion and/or generation of unwanted species. This process would be reversed for negatively charged pharmaceutical compounds. The duration of the anodic or cathodic phases of the electrodes may be of differing duration.

As stated above, control circuit 103 may comprise an embedded processor and program memory to store a control program. The processor and the program memory are integrated into a transdermal patch that can be worn by the user long-term, that is, longer than one week. The control program stored on program memory causes the processor to control the signal generator.

### Processor Control

FIG. 4 illustrates control circuit 103 in more detail, comprising a processor 401, program memory 402, data memory 403 and an electrode port 404. As mentioned above, program code stored on non-transitory, computer readable memory 402 causes the processor 401 to perform the methods disclosed herein. In particular, processor 401 receives measurements from reference electrodes, selects a sub-system and then determines the appropriate control signals to deliver the pharmaceutical. More particularly, processor generates control signals to configure a switching matrix 406 to direct the power signals to the selected sub-system.

Control circuit 103 further comprises a signal driver 405 that comprises the current source as described above. In the example of FIG. 4, there are three data signals and two power signals going from control circuit 103 to switching matrix 406. Two of the three data signals carry a two-bit value to select one of four sub-systems. If the number of sub-systems increases, so do the number of control systems increase to encode the number of sub-systems in a binary number. The third data signal is to receive measurements from the reference electrodes. This data signal may be an analog signal for direct connection to the reference electrode or a binary signal if there is an analog/digital (A/D) converter in the reference electrode.

### User Device

There is also a wireless communication port 407, which sends status updates and receives commands. For example, the communication port 407 may be configured to communicate with a user device 408, such as a patient device or a clinician device. Those devices may be mobile phones/smartphones, and the communication method may be via Bluetooth, Wi-fi or any other communication method. The user device may have an application installed thereon that facilitates monitoring and control of the delivery system 100.

There is a wide range of data that the system 100 may send to the user device. For example, the system 100 may send data indicative of the status of each sub-systems 101, so that the user can see the number of sub-systems 101 that are available. More particularly, the user device 408 may be configured to generate a user interface to show which sub-systems 101 are depleted and which ones are attached properly or have a problem status, such as insufficient attachment.

System 100 may also transmit measurements to user device 408, such as impedance measurements or measurements of the supplied amount of pharmaceutical. Controller 103 may measure an amount of current or charge delivered over time to estimate an amount of the pharmaceutical that has been delivered, where 1 C of measured charge may equate to about 6×10¹⁸ or about 10 μmol of molecules of the pharmaceutical for a particular charge state of the pharmaceutical. User device 408 may store the received data and interpret measurements, may store and manage a pharmaceutical administration profile and patient data. User device 408 may also control the system 100 by selecting sub-systems 101, deactivating sub-systems 101 or by adjusting the delivery of the pharmaceutical. System 100 may also administer the pharmaceutical according to a predetermined time schedule or based on external sensors, such as those integrated into a smart watch or other wearable device.

System 100 may receive an authorisation from user device 408 for the commencement of administration of the pharmaceutical based on patient parameters.

For example, the patient may have an account registered with a health care provider on user device 408. Upon providing credentials into the user device 408, the administration can be started .. The patient parameters may include one or more of patient identity, patient condition, previously administered dose or response to the pharmaceutical or correctly applied patch.

System 100 may further comprise a reverse iontophoresis sub-system that measures a blood concentration of the pharmaceutical or an associated substance that is indicative of the blood concentration of the pharmaceutical. In that case, system 100 can adjust the electrical control system based on the measured blood concentration. This way, the electrical signals, such as pulse amplitude and duration, can be adjusted to maintain a set level of the pharmaceutical in the blood steam. Control circuit 103 may perform a feedback control method to eliminate any error between the set concentration and the measured concentration.

### Electrodes

In one example, the electrodes are coated with an ion-transducing material such as PEDOT:PSS. In that case, the reservoir 202 is considered to be located within the electrode or the reservoir 202 together with the conductor and any coating is referred to as the electrode. Further, the contact between the reservoir and the skin surface 203 is provided by the electrode surface. The skin-electrode interface is made of the ion-transducing material, such that ionised molecules of the pharmaceutical can pass from reservoir 202 into the skin. Further, electrode-skin interface may comprise a hydrogel that is applied onto the electrodes before bringing the electrodes in contact with the skin surface 203. In that case, the electrodes may be coated with materials that increase charge injection into the hydrogel.

Further, the electrodes may be coated with materials that minimise the oxidation or reduction of chemical species that are present in the reservoir. In that sense, the electrodes may be designed to minimise electrolysis of water.

Examples include the use of Polydimethylsiloxane (PDMS), which is an elastomer with excellent optical, electrical and mechanical properties and is biocompatible. Other examples use polyethylene terephthalate (PET). Both PDMS and PET provide for flexible substrates, which is useful as the patch can then adapt to the shape of the skin. Gold (AU), Silver (Ag) and Silver Chloride (AgCl), Iridium Oxide (IrO₂) or Platinum (Pt) can be used for electrodes. Other examples include liquid metal and Ag nanowires for interconnects. Various types of masking materials can be used to define specific areas to hold liquids. Another example includes the use of Poly(3,4-ethylenedioxythiophene) (PEDOT). The electrodes may then be deposited onto that substrate, which may be made of platinum, iridium oxide or another biocompatible conductor.

As set out above, the system 100 may be integrated into a patch comprising an adhesive to affix the patch on the skin of a patient. The patch may integrate all components that are part of the system 100, including control circuit 103, battery 206 and the electrodes.

In some examples, system 100 comprises a polymer on a skin contact surface of the patch. This polymer provides protection of the skin contact surface of the electrodes before application and before opening a package. The polymer is degradable on application to the skin to create a direct contact between the patch and the skin. The polymer skin interface may be a membrane that is permeable to the pharmaceutical.

In one example, the pharmaceutical is dissolved using a solvent and incorporated into a hydrogel that is placed in a well that comprises the electrode 202.

The well may be considered as a type of reservoir for the pharmaceutical. It is noted that different wells may contain different pharmaceuticals. In that case, system 100 may select which of the pharmaceuticals to administer. This may be controllable through user device 408.

The aforementioned hydrogel forms a hydrogel matrix comprising the ionic substance to assist with pharmaceutical transport through the skin. That is, the electrical field generated by delivery electrode 102/201 creates an electrostatic force on the ionic molecules, which pushes the molecules into the skin.

In one example, the well and/or hydrogel matrix incorporates a skin permeator in order to improve the transport of the pharmaceutical through the skin. In one example, the permeator comprises hollow microneedles that puncture the skin and the pharmaceutical can then more readily enter the skin through those punctures.

### Pharmaceutical Substances

The above disclosure mentions ionised pharmaceutical molecules, or salts of pharmaceutical molecules, which means they have a positive or negative charge. In other examples, the pharmaceutical molecules are polar, which means they have a slightly positive end and a slightly negative end. This may still generate sufficient force to push the molecule into the skin.

The proposed solution also works for non-polar pharmaceuticals. In that case, molecules with both polar and non-polar ends are present in the sub-systems and the molecules interact with the non-polar pharmaceutical to increase susceptibility to an electric filed.

In further examples, it may be possible to use ionic tags, which are molecules that are charged and attach to the pharmaceutical molecules. As a result of that, the pharmaceutical comprises nanoparticles that are tagged with an ionic tag and therefore are subject to an electrostatic force.

The pharmaceutical may be included in a nanocarrier, such as polyvinyl alcohol (PVA) or polylactic acid (PLA). This can be considered using a detergent in the liquid where the nanocarriers are located. A detergent has a polar head that attracts oils and has an ionic tail attached to a nanocarrier. As a result, it is possible to perform iontophoresis, which increases the family of drugs where this approach can be used.

It is noted, that in some applications, the drug is made lipophilic so it can diffuse through the skin. However, here the ionic property of the drug is increased since it is to be delivered through the skin. It is noted that the lipophilic property is not significantly influenced by electric field. So in some examples, a detergent is used and the molecule has a lipophilic head and an ionic tail. The application comprises dissolving the pharmaceutical of interest in in a lipid. As a result, the drug is in a lipid shell, which facilitates progression through the skin. The detergent molecules attach and then dangle off the drug molecule so that it has a charge that can be used to create an electrostatic force.

Examples for the pharmaceutical include:
Brivaracetam
Permapanel
Cenobamate
Cannabidiol
Levodopa
Carbidopa
noting that others may equally be suitable for use with the disclosed systems and methods.

### Delivery Method

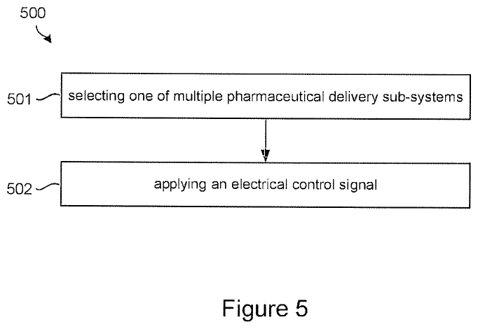

FIG. 5 illustrates a method 500 for delivering a pharmaceutical through iontophoresis. Method 500 comprises selecting 501 one of multiple pharmaceutical delivery sub-systems 101 as described above. Each delivery sub-system 101 comprises a delivery electrode 102. Then, method 500 comprises applying 502 an electrical control signal to the delivery electrode 102 of the selected pharmaceutical delivery sub-system 101 to deliver the pharmaceutical transdermally by iontophoresis.

It is noted that method 500 may be implemented in software in the form of program code executed by a processor. The method may be performed by user device 408 or by control circuit 103 or by a combination of the two. The program code may be stored in non-transitory computer readable medium and cause a processor that executes the program code, to perform method 500.

### Application

This disclosure provides an array-based approach to delivering pharmaceuticals trans-dermally via a wearable patch. Particular aspects of this disclosure includes the use of an array of iontophoresis cells each of which contains a drug mixture and matrix and is independently controlled by a single electrode. Each cell is filled with a specific amount of drug, which enables the precise control of the administration of the drug over an extended period of time. The specific arrangement and geometry of electrodes minimizes the required patch surface area.

As discussed above, the electrode and drug mixture/matrix materials (including coatings) enhance permeation, ensure biocompatibility, facilitate long-term storage of the drug, and minimize potential harm during extended wear. Further, the electrodes can be used as sensors to determine the physical characteristics of the skin and/or other biologic materials. The system-level architecture is chosen to minimize the need for semiconductor components. This may be achieved by using only a single signal generator for all delivery electrodes 102 and connecting that signal generator to the selected electrode.

Further, the electronic waveforms of voltage applied to the electrodes maximizes drug delivery, minimizes the creation of deleterious chemical species, and minimizes irritation and/or damage to the skin. The encapsulation methodology includes the use of nano-carriers to make specific drugs compatible with iontophoresis-based drug delivery. The physical architecture of the patch includes specific adhesives, methods to store drug matrices, bio-resolvable layers, and wireless communication and measurement apparatus. Further, system-level programming techniques ensure the consistent delivery of drugs through the skin to the bloodstream. Finally, the disclosed systems and methods can be used in the application of the delivery of drugs for epilepsy treatment. This treatment is specifically well suited to the disclosed solutions because epilepsy drugs are best administered on a continuous basis and the overall amount of the pharmaceutical is not too large for a transdermal application.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1.-39. (canceled)

40. An iontophoresis pharmaceutical delivery system, comprising:
(a) two or more individually addressable pharmaceutical delivery sub-systems, each of said delivery sub-systems comprising
a. a reservoir comprising a pharmaceutically active agent incorporated into a hydrogel matrix, and
b. a delivery electrode that is insulated from electrodes of other delivery sub-systems; and
(b) a control circuit configured to generate an electrical control signal and provide the electrical control signal to the delivery electrode of one or more of said delivery sub-systems to deliver said pharmaceutically active agent;
wherein said delivery system comprises two or more pharmaceutically active agents and is configured to select and deliver one or more of said pharmaceutically active agents.

41. An iontophoresis pharmaceutical delivery system according to claim 40, wherein said delivery system is integrated into an adhesive patch for transdermal application

42. An iontophoresis pharmaceutical delivery system according to claim 41 wherein said control circuit comprises a wireless communication port configured to communicate with a user device.

43. An iontophoresis pharmaceutical delivery system according to claim 42, wherein said user device is a smartphone comprising an application installed thereon that facilitates monitoring and control of the delivery system.

44. An iontophoresis pharmaceutical delivery system according to claim 42, wherein said control circuit is configured to transmit data to said user device.

45. An iontophoresis pharmaceutical delivery system according to claim 44, wherein said data is indicative of the status of one or more of said delivery sub-systems.

46. An iontophoresis pharmaceutical delivery system according to claim 45, wherein said data is indicative of the amount delivered of said one or more pharmaceutically active ingredients.

47. An iontophoresis pharmaceutical delivery system according to claim 46, wherein said data is indicative of the amount of current or charge delivered over time.

48. An iontophoresis pharmaceutical delivery system according to claim 46, wherein said user device is configured to store said data.

49. An iontophoresis pharmaceutical delivery system according to claim 44, wherein user device is configured to interpret said data and create a pharmaceutical administration profile.

50. An iontophoresis pharmaceutical delivery system according to claim 42, wherein said user device communicates authorization for said electrical control signal to said control circuit.

51. An iontophoresis pharmaceutical delivery system according to claim 41, wherein said pharmaceutically active agent is selected from the group consisting of brivaracetam, perampanel, cenobamate, cannabidiol, levodopa, and carbidopa.

52. An iontophoresis pharmaceutical delivery system according to claim 41, wherein said pharmaceutically active agent is included in a nanocarrier.

53. An iontophoresis pharmaceutical delivery system according to claim 52, wherein said nanocarrier comprises polyvinyl alcohol or polylactic acid.

54. An iontophoresis pharmaceutical delivery system according to claim 41, wherein said pharmaceutically active agent is conjugated to a charged molecule.

55. An iontophoresis pharmaceutical delivery system according to claim 41, wherein said hydrogel matrix comprises an ionic transporter.

56. An iontophoresis pharmaceutical delivery system according to claim 41, wherein said hydrogel matrix comprises a skin permeator.
